(19) United States
(12) Patent Application Publication
(10) Pub. No.: US 2013/0325067 A1
(43) Pub. Date: Dec. 5, 2013

(54) INTERVERTEBRAL DISTRACTOR

(71) Applicants: Giuseppe Calvosa, Pisa (PI) (IT); Raphael Bartalesi, Firenze (FI) (IT); Miria Tenucci, Lucca (LU) (IT)

(72) Inventors: Giuseppe Calvosa, Pisa (PI) (IT); Raphael Bartalesi, Firenze (FI) (IT); Miria Tenucci, Lucca (LU) (IT)

(21) Appl. No.: 13/988,564

(22) PCT Filed: Nov. 23, 2010
(86) PCT No.: PCT/IB10/55377

§ 371 Date: Aug. 21, 2013

(51) Int. Cl. A61B 17/70

(52) U.S. Cl. CPC A61B 17/7065
USPC 606/249

(57) ABSTRACT

An interspinous intervertebral distractor is described. The intervertebral distractor has a main body to be inserted between two adjacent spinous processes to provide a related support, a first and a second pair of lateral stabilizers, arranged respectively in correspondence of a first and a second end of the main body, and a rod that is percutaneously actuatable and has a shaped profile to cause the spreading apart of the stabilizers of the first pair, and of the second pair.

The present invention refers to an intervertebral distractor of interspinous type suitable for a percutaneous insertion, and in particular to a distractor of the type comprising a double pair of lateral stabilizers, applied on a main body and selectively spreadable apart with respect to the latter to hold the body itself in position between two adjacent spinous processes.

Intervertebral distractors are devices apt to space apart two adjacent vertebrae. In particular, the distractors subject-matter of the present invention are prostheses conceived to be permanently implanted in the space comprised between the spinous processes of adjacent vertebrae, in order to maintain an intervertebral distraction such as to limit the loads transmitted between said vertebrae by effect, e.g., of degenerative pathologies of the intervertebral discs, and to contain the associated painful manifestations.

With respect to other vertebral prostheses, the interspinous distractors can be easily inserted in their seat, thanks to the relative ease with which the spinous processes of two adjacent vertebrae allow slight spreading apart. For the same reason, such distractors do not compromise local mobility of the rachis in flexion, but reduce hyperextension thereof.

Against such advantages, however, known stabilization problems subsist. In fact, the distractor has to be maintained in position, in particular has to be constrained with respect to displacements such as to compromise its functionality or even cause its ejection from the seat, with movements in the frontal plane of the patient. Such a stabilization function is generally carried out by lateral fins of the distractor, apt to abut on the spinous apophyses.

However, known distractors suffer from some relevant drawbacks and problems still unsolved. In particular, the need to spread apart in situ said fins has entailed the implementation of systems that are mechanically complex, and therefore often lack structural strength, generally making the distractors scarcely reliable.

Such a drawback constitutes a relevant restriction, also to the ends of a miniaturization of the distractor, e.g. in order to adapt to individuals with a very small frame or of infant age.

Moreover, a further problem is represented by the instruments required to perform such a spreading apart, them also often complex and rather complicated to handle percutaneously, to the detriment of the rapidity of surgery.

Therefore, the technical problem set and solved by the present invention is that of providing an intervertebral distractor of the above-mentioned type, allowing to overcome the drawbacks mentioned above with reference to the known art.

Such a problem is solved by an intervertebral distractor according to claim 1.

Preferred features of the present invention are set forth in the dependent claims thereof.

The present invention provides some relevant advantages. One of the main advantages lies in the fact that in the distractor of the invention the means for spreading apart the fins exhibit an implementation mechanically reliable and sturdy at the same time, and are based on a single elongated element allowing the independent spreading apart of the two pairs of stabilizers.

Moreover, such spreading apart means (retractors) is actuatable from the outside, i.e. percutaneously, in a simple and effective manner.

Other advantages, features, and the operation steps of the present invention will be made apparent in the following detailed description of some embodiments thereof, given by way of example and not for limitative purposes. Reference will be made to the figures of the annexed drawings, wherein:

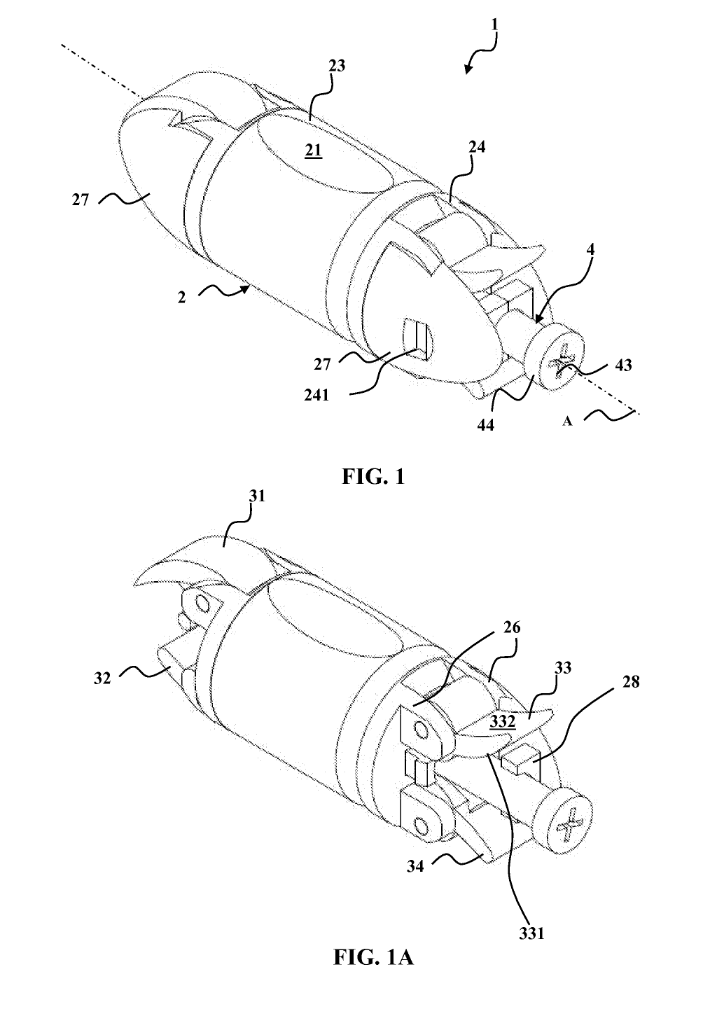

FIG. 1 shows a perspective view of a preferred embodiment of the intervertebral distractor of the invention, in a first inserting configuration for insertion into a patient's body, in which the lateral stabilizers are closed;

FIG. 1A shows a perspective view of the distractor of FIG. 1, in which two lateral protective shells have been removed for greater clarity;

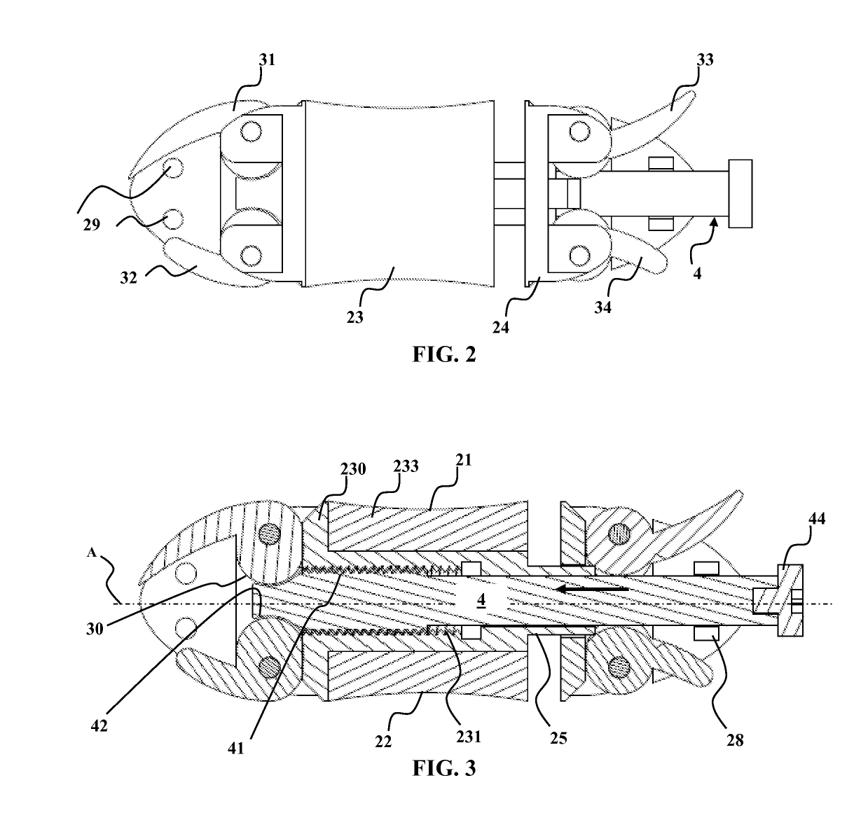

FIG. 2 shows a side view of the distractor of FIG. 1, again with two lateral shells removed;

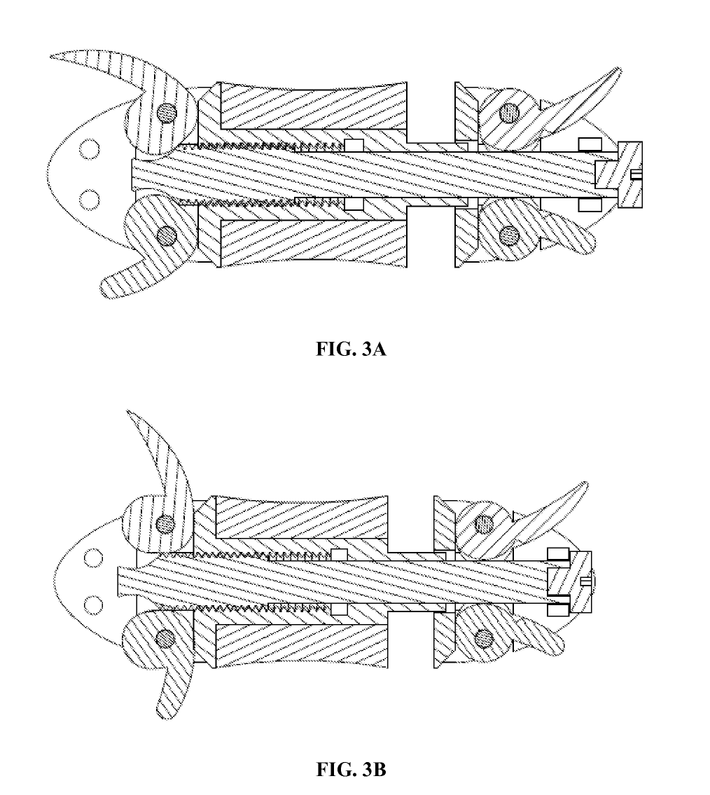

FIG. 3 shows a longitudinal sectional view of the distractor of FIG. 1;

FIGS. 3A and 3B show each another longitudinal sectional view of the distractor of FIG. 1, in a respective intermediate configuration for opening the distal stabilizers with respect to the surgeon;

FIG. 4 shows a perspective view of the distractor of FIG. 1, in a second configuration for holding in situ into a patient's body, in which the lateral stabilizers are spread apart;

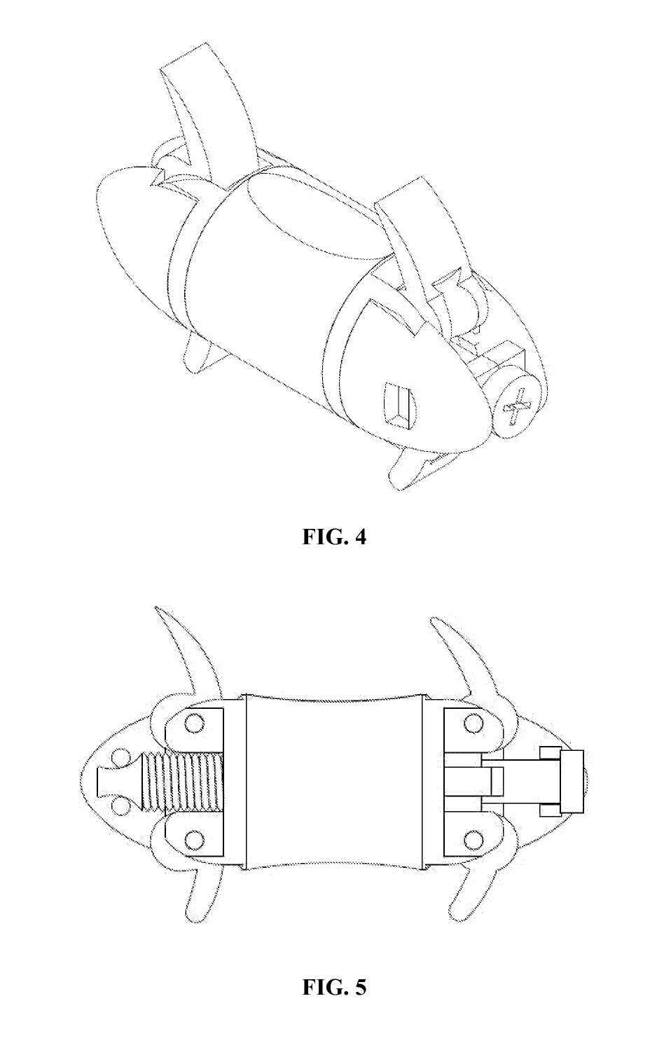

FIG. 5 shows a side view of the distractor of FIG. 1, in the configuration of FIG. 4, with two-lateral shells removed;

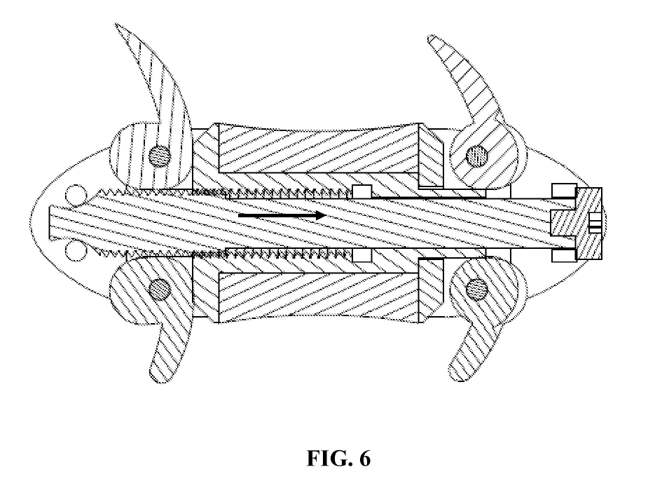

FIG. 6 shows a longitudinal sectional view of the distractor of FIG. 1, in the configuration of FIG. 4;

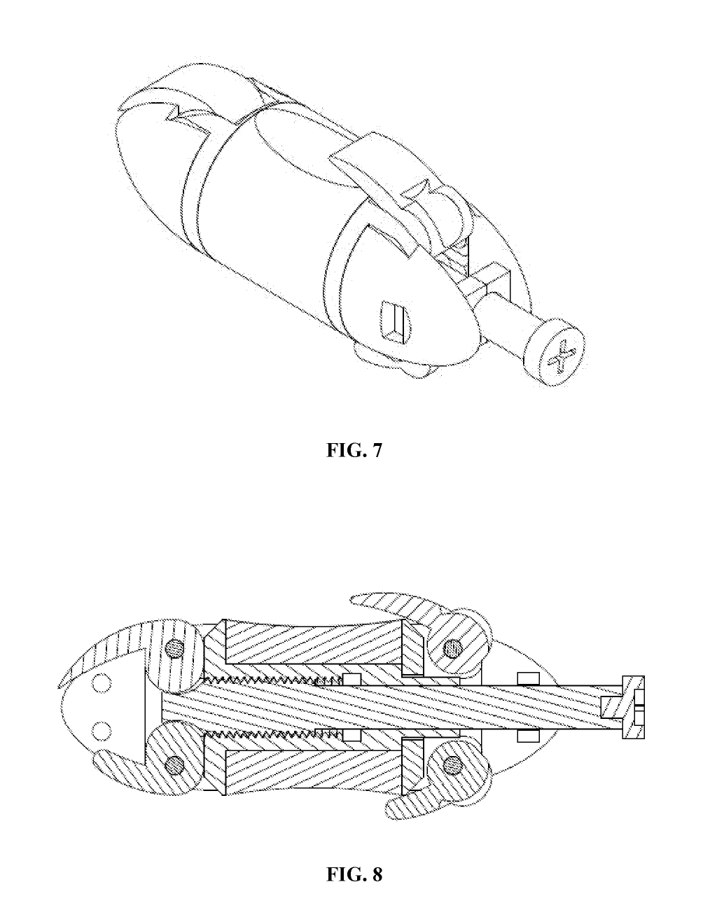

FIG. 7 shows a perspective view of a variant embodiment of the distractor of the invention, in a further extracting configuration in which the lateral stabilizers arranged proximally with respect to the surgeon's position are further spread apart, and those arranged distally are closed; and

FIG. 8 shows a longitudinal sectional view of the distractor of FIG. 7.

Referring initially to FIGS. 1, 1A, 2 and 3, an intervertebral distractor of interspinous type according to a preferred embodiment of the invention is generally denoted by 1.

The distractor 1 has a generally tapered configuration, fostering a percutaneous insertion thereof.

The distractor 1 comprises first of all a main body 2, apt to be inserted between two adjacent spinous processes to provide a related support. In the present embodiment, the body 2 has a substantially elongated configuration with generally substantially elliptic cross sections. The body 2 bears, topwise and bottomwise, a respective depression or concavity 21, 22, substantially forming a saddle, apt to foster its own stabilization in situ between two spinous processes. It will be understood that in the present context the definitions of “top” and “bottom” are referred to the position of the distractor 1 when used in situ with the subject in standing position.

The main body 2 has a longitudinal axis denoted by A.

The main body 2 is implemented in two portions, respectively 23 and 24, slidably coupled to each other according to modes that will be detailed hereinafter. For greater clarity, such two portions 23 and 24 will be denoted respectively as first and second portion, or respectively as distal and proximal portion. These latter two terms are to be understood, here and to hereinafter, as referred to the position of the distractor 1 with respect to the surgeon during insertion into a patient's body.

The first portion 23, of greater extension, has a tubular prolongation, or appendage 25, with a substantially cylindrical development, extending symmetrically along the longitudinal axis A and engaging a corresponding seat of the second portion 24.

As will be illustrated hereinafter, such tubular appendage 25 implements an abutment means against the retractors that will be described hereinafter.

To the second portion 24 of the body 2 it is also integrally associated a substantially C-shaped member 28, implementing a means for stopping a rod of the distractor, rod which will it also be introduced hereinafter.

Moreover, the main body 2 bears, in correspondence of or near to a first longitudinal end thereof associated to the above-mentioned first portion 23, two pairs of flanges 26 for coupling with lateral stabilizers that will be described below. An analogous double pair of flanges 26 is provided in correspondence of or near to the second longitudinal end of the body 2 associated to the second portion 24 thereof.

Moreover, in correspondence of each of said first and second end, the main body 2 has a pair of protective shells transversally placed side by side, denoted by 27, which have a configuration tapered in the direction of the longitudinal axis A, thereby fostering the percutaneous insertion of the distractor 1.

The first portion 23 of the body 2 bears also a pair of pins 29 integral to one of said shells 27 and internally projecting therefrom, the function of which will be made clear hereinafter.

As seen, e.g., in FIG. 2, in the present embodiment the first portion 23 is in turn formed by a frame 230 bearing the above-mentioned appendage 25, the shells 27, the pins 29 and the flanges 26, and by a distractor body 233 bearing the saddles 21 and 22, arranged externally to the frame 230 itself and integral therewith. Preferably, the frame 230 is made of Titanium, whereas the distractor body 233 is made of PEEK.

The distractor 1 further comprises a first—distal—pair of stabilizers, denoted by 31 and 32, and a second—proximal—pair of stabilizers, denoted by 33 e 34, associated respectively to said first and second portion 23, 24 of the to distractor 1. In particular, each stabilizer 31-34 is rotatably connected to the respective portion 23, 24 of distractor in correspondence of a flange 26 thereof. More specifically, each stabilizer 31-34 is hinged on said flange in correspondence of an end thereof.

Each stabilizer 31-34 has a substantially elongated, curved fin-like configuration, with a first convex profile and a corresponding concave profile. By way of example, the convex profile of the proximal stabilizer 33 is denoted by 331, and the concave one by 332.

The rotatable connection with the main body 2 enables the stabilizers 31-34 of each pair to rotate between a first closed position, shown in FIG. 1, in which they implement a minimal encumbrance configuration, fostering a percutaneous insertion of the distractor 1 into a patient's body, and a second opened configuration, shown in FIG. 4, in which they are spread apart so as to be able to engage from a respective side the spinous apophyses, holding in situ the main body 2.

As shown in FIGS. 1, 1A, 2 and 3, the overall arrangement is such that in the closed configuration the stabilizers 31 and 32 of the first pair have their own concave profile facing towards the main body 2, i.e. giving onto axis A, and the stabilizers 33 and 34 of the second pair have instead their own convex profile in such position.

On the contrary, in the spread apart configuration of FIGS. 4, 5 and 6, the stabilizers 33 and 34 of the second pair have, in general, their own concave profile facing towards the main body 2, i.e. giving onto axis A.

As shown in the drawings, preferably the bottom stabilizers 32 and 34 of the two pairs have an extension generally lower than the corresponding top stabilizers 31 and 33 of the same pair. This contrivance allows to prevent interferences of the distractor 1 with the laminae and/or the articular facets of the lower vertebra concerned by the distractor itself. This is particularly useful in case of bone hypertrophies or deformations, scoliosis, and in the case of vertebral levels comprised in the range L5-S1.

The distractor 1 further comprises an elongated element, or rod, generally denoted by 4, percutaneously actuatable to cause the passage of the stabilizers 31-34 from the first to the second configuration illustrated above.

Such rod 4 is housed within the main body 2, and in particular within the frame 230, in correspondence of the longitudinal axis A and slidably coupled to such body 2, so that the related movement be carried out precisely along such axis A. In particular, the rod 4 extends within the first and the second portion 23 and 24 of the body 2, engaging also the above-mentioned tubular appendage 25 of the first portion 23.

In the present example, the slidable coupling is of screw-nut screw threaded type, the rod 4 bearing, in correspondence of its own distal portion, a threading 41 complementary to a corresponding nut screw threading 231 obtained internally to the first portion 23.

The rod 4 further has, in correspondence of a distal end thereof, spreading means for spreading apart the stabilizers 31 and 32 of the first pair, which in the present embodiment are implemented by a shaped profile 42 apt to form a shape coupling with such stabilizers 31 and 32 so as to cause precisely, when needed, the spreading apart.

Always in the present example, the shaped profile 42 of the rod 4 is of cam type. In particular, the shaped profile 42 is of concave type and the stabilizers 31 and 32 of the first pair have, in correspondence of the rotatable connection to the main body 2, a corresponding convex profile 30 conjugated with the concave profile 42.

Moreover, the rod 4 has, substantially oppositely to the shaped profile 42, means for coupling with a percutaneously actuatable manipulation instrument. In the present example, such means is implemented by a further shaped profile 43, obtained on a head 44 and apt to be engaged by a screwdriver instrument. To a technician in the field it will be obvious that the end 44 may have other forms of coupling for the actuation instrument.

The rod 4 may also be made hollow in order to allow, e.g., the insertion of a guide wire or the like.

The above-mentioned member 28 also increases the rigidity of the rod 4, limiting its flexure.

Hereinafter, the operation of the distractor 1 will be described with regard to the passage of the stabilizers 31-34 from the first to the second position illustrated above.

At the percutaneous insertion of the distractor 1, the latter appears in closed configuration, as shown in FIGS. 1, 1A, 2 and 3. The stabilizers are therefore rotated into a minimal encumbrance position, with their own convex/concave profiles arranged as already described above.

The rod 4 appears in a position maximally set back in the proximal direction. In particular, its concave profile 42 engages the complementary convex profile 30 of the stabilizers 31 and 32 of the first pair, so that said stabilizers may assume the above-mentioned closed position. In the maximally set back position, the concave profile 42 is such as to oppose to an accidental opening up of the stabilizers 31 and 32, opening up that the tissues might cause during an insertion of the device.

The percutaneous insertion of the distractor can occur by cannula and/or guide wires according to procedures already known to the technician in the field, or to innovative procedures subject of a separate patent application.

During such insertion, the main body 2 can be held by engagement of a dedicated instrument into suitable lateral seats of the second portion 24, one of which is exemplarily denoted by 241 in FIG. 1. Concomitantly, the advancement of the distractor may be obtained by acting with a conventional instrument on the head 44 of the rod 4.

Once completed the actual inserting step, the main body 2 is housed between two adjacent spinous processes. Then, the rod 4 is percutaneously actuated by a screwdriver instrument in order to produce the selective spreading apart of the stabilizers 31-34, and this according to the procedure illustrated hereinafter.

First of all, the rod 4 is slid along the longitudinal axis A of the main body 2 in a distal direction, as per arrow reported in FIG. 3. In such movement, the rod slides with respect to both portions 23 and 24 of the main body 2, which therefore remains stationary. Following such sliding, the concave profile 42 of the rod 4 engages the corresponding convex profile 30 of the latter stabilizers 31 and 32 so as to cause their spreading apart, as shown in FIG. 3A.

The further sliding therefore causes the full spreading apart of the stabilizers 31 and 32, shown in FIGS. 4 to 6. In such a spread apart configuration, such stabilizers are externally abutted on the lateral spinous apophyses, preventing movement of the distractor 1 in the proximal direction. As shown in FIGS. 5 and 6, further distal movement of the rod 4 is prevented, always in the configuration considered herein, by the engagement of the concave profile 42 thereof with the pins 29, implementing therefore means for the distal stopping of the rod itself.

Always in the fully spread apart configuration of the stabilizers 31 and 32, the head 44 of the rod 4 is abutted against the stopping means 28 associated to the second portion 24 of the body 2. Such means 28 is therefore it also apt to cause the stopping of the distal movement of the rod 4.

In the configuration presently considered, a further rotation of the screwdriver instrument engaging the rod 4 causes a reversion of the related motion, in the sense of producing the sliding in the proximal direction of the first portion 23 of the main body 2 with respect to the rod 4 itself and to the second portion 24, as indicated by the arrow reported in FIG. 6. Therefore, such proximal sliding causes the approaching of the tubular appendage 25 to the stabilizers 33 and 34 of the second pair, the subsequent abutting of the former on corresponding convex profiles of the latter, and then the spreading apart thereof shown in FIGS. 4 to 6.

In the completely spread apart position, the first and the second portion 23 and 24 of the main body are abutted against each other, forming one body.

Upon reaching such spreading apart, the stabilizers 33 and 34 of the second pair are abutted on the spinous apophyses contralateral with respect to those of the first pair 31, 32.

Incidentally, it has to be noted that during the insertion of the distractor and the opening up of the distal stabilizers 31 and 32 it is not possible to accidentally open the proximal ones 33 and 34, as these are retained by the cannulation. Only when the distal stabilizers 31 and 32 have opened up, the cannula is slightly unthreaded and the opening up of the proximal stabilizers is allowed.

By now, it will be better appreciated that the rod 4 allows a reversible spreading apart of the stabilizers 31-34, in the sense of allowing, by reversing the hereto-described procedure, the reclosing thereof.

It will also be appreciated that the arrangement described allows an independent spreading of the stabilizers of the first pair with respect to those of the second pair (and vice versa).

Moreover, it should be noted that the arrangement described allows a continuous change of the position of the stabilizers of the two pairs.

It will also be appreciated that a single instrument, in the case considered herein a screwdriver, is required to perform the spreading apart of both pairs of stabilizers.

Furthermore, the technician in the field will appreciate that it is possible to provide, in combination with the hereto-described components of the distractor, also specific means for holding one or more stabilizers in the above-described extreme spread apart and closed positions, means that can be disengaged when needed.

Referring to FIGS. 7 and 8, on the basis of a variant embodiment it is provided that the stabilizers 33 and 34 of the second pair may rotate with respect to the main body 2 of an angle greater than about 90 degrees, and in particular preferably of an angle comprised in a range of about 120-180 degrees.

Such increased angle is advantageous since it is possible, when it is necessary to extract the distractor 1, to bring said stabilizers 33 and 34 into a further minimal encumbrance configuration in which they are substantially “upturned” on the distractor body 233 and expose their convex profile so as to facilitate the extraction itself.

Therefore, overall the stabilizers of the first pair may be continuously rotated among three reference positions; specifically, a closed position of insertion, a spread apart position of holding in situ and a further spread apart extracting position. The stabilizers of the first pair can be rotated between the first two positions mentioned above.

The present invention has been hereto described with reference to preferred embodiments thereof. It is understood that other embodiments might exist, all falling within the concept of the same invention, as defined by the protective scope of the claims hereinafter.

1. An interspinous intervertebral distractor, comprising:
a distraction main body, adapted to be inserted between two adjacent spinous processes to provide support;
a first and a second pair of lateral stabilizers, arranged respectively in correspondence of or near to a first and a second end of said distraction main body, each stabilizer of said first and second pair of lateral stabilizers being rotatably connected to said distraction main body and movable between a first closed position, in which the stabilizers of the first and second pair of lateral stabilizers implement a minimal encumbrance configuration, fostering a percutaneous insertion of the intervertebral distractor into a patient's body, and a second opened configuration, in which the stabilizers of the first and second pair of lateral stabilizers are spread apart so as to be able to engage from a respective side the spinous apophyses, holding in position said distraction main body; and
an elongated element slidably coupled with said distraction main body, said elongated element being percutaneously actuatable and having spreading means adapted to cause, when needed, a spreading apart of the first pair of lateral stabilizers,
wherein said distraction main body comprises a first and a second portion, slidably coupled and bearing respectively said first and second pair of lateral stabilizers, wherein the percutaneous actuation of said elongated element causes a relative sliding between said first and second portion, which in turn produces a spreading apart of the second pair of lateral stabilizers.

2. The intervertebral distractor according to claim 1, wherein said elongated element is movable within said distraction main body along a longitudinal axis of said distraction main body.

3. The intervertebral distractor according to claim 1, wherein said elongated element has a screw coupling with said first portion of said distraction main body.

4. The intervertebral distractor according to claim 1, wherein said first portion of said distraction main body has an appendage adapted to cause, following said relative sliding between said first and second portion, the spreading apart of the second pair of lateral stabilizers.

5. The intervertebral distractor according to claim 1, wherein said second portion of said distraction main body has means for stopping said elongated element, adapted to prevent the relative sliding of the latter towards said second portion.

6. The intervertebral distractor according to claim 1, wherein said first portion of said distraction main body has means for stopping said elongated element, adapted to prevent the relative sliding of the latter towards said second portion.

7. The intervertebral distractor according to claim 1, wherein said spreading means comprises a shaped profile adapted to form a shape coupling with the first pair of lateral stabilizers.

8. The intervertebral distractor according to claim 7, wherein said shaped profile of said elongated element is a cam.

9. The intervertebral distractor according to claim 7, wherein said shaped profile of said elongated element is a concave profile and the first pair of lateral stabilizers have a corresponding convex profile conjugated with said concave profile.

10. The intervertebral distractor according to claim 1, wherein said elongated element has, substantially oppositely to said spreading means, means for coupling with a percutaneously actuatable manipulation instrument.

11. The intervertebral distractor according to claim 10, wherein said means for coupling means comprises a shaped profile adapted to be engaged by a screwdriver instrument.

12. The intervertebral distractor according to claim 1, wherein the first pair of lateral stabilizers have each a concave profile that is facing, in a closed position, towards said distraction main body.

13. The intervertebral distractor according to claim 1, wherein the second pair of lateral stabilizers have each a convex profile that is facing, in a closed position, towards said distraction main body.

14. The intervertebral distractor according to claim 1, comprising means for stopping the first pair of lateral stabilizers and/or the second pair of lateral stabilizers in a closed position.

15. The intervertebral distractor according to claim 1, wherein the second pair of lateral stabilizers is adapted to assume a third, further spread apart, extracting position.

16. The intervertebral distractor according to claim 15, wherein the second pair of lateral stabilizers is adapted to rotate with respect to said distraction main body of an angle greater than about 90 degrees, so as to take a further minimal encumbrance configuration.

17. The intervertebral distractor according to claim 16, wherein an angle of rotation is comprised in a range of 120-180 degrees.

18. The intervertebral distractor according to claim 1, wherein said elongated element is slidable in two directions, so as to be percutaneously actuatable to cause a passage of the first and second pair of lateral stabilizers from said second position to said first position.

19. The intervertebral distractor according to claim 1, wherein the first and second pair of lateral stabilizers are hinged on said distraction main body in correspondence of respective ends.

20. The intervertebral distractor according to claim 1, wherein an overall arrangement is such as to cause a continuous change of a position of the first and second pair of lateral stabilizers.

21. The intervertebral distractor according to claim 1, wherein an overall arrangement is such as to cause an independent spreading of the stabilizers respectively of said first and second pair of lateral stabilizers.

22. The intervertebral distractor according to claim 1, wherein a stabilizer of each of said first and second pair of lateral stabilizers has extension lower than the other stabilizer of the same pair of stabilizers of the first and second pair of lateral stabilizers.
